(19) United States
(12) Patent Application Publication
(10) Pub. No.: US 2024/0068025 A1
(43) Pub. Date: Feb. 29, 2024

(54) GENOMIC ANALYSIS METHOD

(71) Applicant: Perseus Biomics BV, Tienen (BE)

(72) Inventor: Volker Leen, Kortenaken (BE)

(21) Appl. No.: 18/267,180

(22) PCT Filed: Dec. 22, 2021
(86) PCT No.: PCT/EP2021/087261

§ 371 Date: Jun. 14, 2023

(30) Foreign Application Priority Data
Dec. 22, 2020 (EP) 20216370.5

(51) Int. Cl. C12Q 1/6874

(52) U.S. Cl. CPC C12Q 1/6874

(57) ABSTRACT

The present invention relates to and includes methods and compositions for sequence-specific labeling of DNA, in particular genomic DNA. Such labeling result from the application of agents that covalently bind or interact with predetermined target nucleic acid sequences within the DNA, enabling detecting a relative distance between the labels on the linearized DNA, thus providing a barcode of a portion of the genomic DNA, and the use thereof for the analysis of genomic DNA. Preferably, the covalently binding or interaction is with the grooves of double-stranded DNA (dsDNA). In some embodiments, the analysis of genomic DNA according to the invention can be used for species identification, where these species are single species, or identified in mixtures of species, as to identify the presence of species or the composition of the mixture of species.

### TECHNICAL FIELD OF THE INVENTION

Embodiments herein relate generally to labeling DNA molecules, for example genomic labeling for analysis of linearized DNA.

The present invention in particular relates to and includes methods and compositions for sequence-specific labeling of DNA, in particular genomic DNA. Such labeling result from the application of agents that covalently bind or interact with predetermined target nucleic acid sequences within the DNA, enabling detecting a relative distance between the labels on the linearized DNA, thus providing a barcode of a portion of the genomic DNA, and the use thereof for the analysis of genomic DNA. Preferably, the covalently binding or interaction is with the grooves of double-stranded DNA (dsDNA). In some embodiments, the analysis of genomic DNA according to the invention can be used for species identification, where these species are single species, or identified in mixtures of species, as to identify the presence of species or the composition of the mixture of species.

### BACKGROUND OF THE INVENTION

High throughput DNA sequencing technologies have sparked a revolution that will radically transform biological and biomedical research. It is increasingly realized that many biological and biomedical problems can and only be addressed through large scale sequencing of DNA or RNA. For example, through large scale sequencing, we can rapidly grasp the scale of mutations in cancers. Large scale and cost effective sequencing also makes previously difficult endeavors straightforward. For example, identification of a disease gene in a large genomic region can now be directly tackled by targeted DNA sequencing of the region harboring the disease gene. As these high throughput analysis technologies become increasingly accessible to researchers, they are frequently used to address previously impossible problems.

However, broad applications of these technologies are still limited by their high costs in both equipment acquisition and reagent consumption. The cost of resequencing a mammalian-sized still remains in the range thousands of dollars, which is far too high for many applications that require sequencing of a large number of samples. Additionally, some of the major challenges in genome analysis are de novo genome sequence assembly based on ‘short read’ shotgun sequencing and structural variation analysis. Several approaches and combinations of different approaches have been attempted to meet these challenges. The most widely adopted strategy relies on deep sequencing of shotgun libraries and sequencing of mate-pair libraries, which increases the sequence contiguity of short-read sequencing (See, Siegel, A. F., et al. (2000) “Modeling the feasibility of whole genome shotgun sequencing using a pairwise end strategy.” Genomics 68(3): 237-246). Another approach relies on the stochastic separation of corresponding genomic or polymerase chain reaction (PCR) fragments into physically distinct pools followed by subsequent fragmentation to generate shorter sequencing templates (See, Kaper, F., et al. (2013). “Whole-genome haplotyping by dilution, amplification, and sequencing.” Proceedings of the National Academy of Sciences of the United States of America 110(14): 5552-5557; Kuleshov, V., et al. (2014) “Whole-genome haplotyping using long reads and statistical methods.” Nature Biotechnology 32(3): 261-266. Additionally, longer-read sequencing technologies such as PacBio®'s SMRT and Oxford Nanopore sequencing promise to eventually further improve assembly contiguity. For example, SMRT sequencing has been successfully applied to closing some gaps and detecting some structural variations in the human reference genome (For example, See Chaisson, M. J. P., et al. (2015) “Resolving the complexity of the human genome using single-molecule sequencing.” Nature 517(7536): 608-611). However, their high error rate, low throughput and high cost have thus far prevented widespread adoption.

None of the aforementioned approaches, however, adequately address the problems of long-range de novo assembly contiguity and validation, sequence mis-assembly in complex regions or accurate assignment of species identity in complex mixtures or metagenomes. Whole genome mapping technologies can provide complementary tools, offering scaffolds for genome assembly, structural variation analysis or high-information species recognition in microbiomes. DNA mapping, pioneered by David Schwartz and colleagues in the form of optical mapping, has been used to construct restriction maps for various genomes and has proven to be very useful in providing scaffolds for shotgun sequence assembly and detection of structural variations (See, Samad, A., et al. (1995) “Optical Mapping—A novel, single-molecule approach to genomic analysis.” Genome Research 5(1): 1-4; and Teague, B., et al. (2010) “High-resolution human genome structure by single-molecule analysis.” Proceedings of the National Academy of Sciences of the United States of America 107(24): 10848-10853). Furthermore, Ming Xiao and colleagues developed a highly-automated whole genome mapping in a nanochannel array (Hastie, A. R., et al. (2013). “Rapid Genome Mapping in Nanochannel Arrays for Highly Complete and Accurate De Novo Sequence Assembly of the Complex Aegilops tauschii Genome.” Plos One 8(2); Lam, E. T., et al. (2012) “Genome mapping on nanochannel arrays for structural variation analysis and sequence assembly.” Nature Biotechnology 30(8): 771-776 and US 2016/0168621 A1.

The above-described genome mapping strategies are based on mapping the distribution. Labels are incorporated at these sequence motifs using complex and cumbersome methods. Often, these labels are short (from 4 bp to 8 bp) sequence motifs across the genome However, the distribution of the sequence motifs is uneven at different genomic regions, which can lead to variation in sequence specificity and this signal quality. Often, large amounts of reagents are leading to cross reactivity and spurious labeling of enzymes. Hence, it is desirable to obtain new methods that accurately address DNA labeling of basepair sequence motifs.

### SUMMARY OF INVENTION

The present invention relates to and includes methods and compositions for sequence-specific labeling of polynucleotides, in particular genomic DNA. Such labeling result from the application of agents that γ bind or interact with predetermined target nucleic acid sequences within the DNA, followed by covalent attachment of a label at or near the predetermined target nucleic acid sequence, thus enabling detecting a relative distance between the labels or the sequence of the labels on the linearized DNA, thus providing a barcode of a portion of the genomic DNA, and the use thereof for the analysis of genomic DNA. Preferably, the covalently binding or interaction is with the grooves of double-stranded DNA (dsDNA). In some embodiments, the analysis of genomic DNA according to the invention can be used for species identification, where these species are single species, or identified in mixtures of species, as to identify the presence of species or the composition of the mixture of species.

Other aspects of the invention will be apparent from the description and examples below, and can be summarized according to the following numbered embodiments.

1. A genomic analysis method, comprising;
a. Subjecting a polynucleotide to a covalent sequence specific labeling,
b. Linearizing said sequence specific labeled polynucleotide, and
c. Obtaining positional information on the sequence specific labels
   2. The genomic analysis method according to embodiment 1, wherein the step of subjecting the polynucleotide to a covalent sequence specific labeling, comprises contacting said polynucleotide with a specific labeling agent comprising a portion, e.g. a binding sequence or sequence specific structure, complementary to a target sequence in the polynucleotide, and wherein the specific labeling agent is configured to bind a label on the polynucleotide at a location within or adjacent to the target sequence.
   3. The genomic analysis method according to embodiment 2, wherein the specific labeling agent comprises a moiety capable of recognizing specific sequences of nucleic acids or abundances of nucleic acids or nucleic acid combinations
   4. The genomic analyis method according to embodiment 2, wherein the specific labeling agent contains a reactive group which can react covalently with the polynucleotide within or adjacent to the target sequence.
   5. The genomic analysis method according to embodiment 2, wherein the specific labeling agent comprises a label or a reactive labeling group which can react with a label after covalent attachment of the specific labeling agent to the polynucleotide.
   6. The genomic analysis method according to embodiment 2, wherein the binding sequence or sequence specific structure is selected from the group comprising: benzimidazole dimers and oligomers, pyrrole oligomers, flavones, pyrrole-imidazole oligoamides, synthetic oligodeoxynucleotides (ODN), triple-helix forming oligonucleotides, or a combination thereof
   7. The genomic analysis method according to embodiment 3, wherein the reactive group is selected from the group comprising: platinum complexes, electrophiles (such as mustards, aziridines), nitrenes, carbenes, and the like.
   8. The genomic analysis method according to embodiment 4, wherein the label is selected from the group comprising, a fluorophore, a quantum dot, a dendrimer, a nanowire, a bead, a hapten, a streptavidin, an avidin, a neutravidin, a biotin, a reactive group, a peptide, a protein, a magnetic bead, a radiolabel, a non-optical label, or a combination of two or more of the listed items.
   9. The genomic analysis method according to embodiment 5, wherein the reactive labeling groups are bioorthogonal in reactivity.
   10. The genomic analysis method as herein provided, wherein the step of linearizing said sequence specific labeled polynucleotide, comprises linearizing the labeled polynucleotide in a fluidic channel, on a surface, or through a nanopore.
   11. The genomic analysis method according to embodiment 2 , wherein the polynucleotide is contacted with multiple sequence specific labeling agents, each agent having a portion complementary to a different target sequence in the polynucleotide.
   12. The genomic analysis methods according to the present invention wherein the polynucleotide is selected from the list comprising: genomic DNA, plasmid DNA, mRNA, tRNA and genomic RNA; in particular genomic DNA.
   13. Use of the genomic analysis methods according to the present invention in providing a barcode of a portion of genomic DNA.

### BRIEF DESCRIPTION OF THE DRAWINGS

FIG. 1 is a schematic depiction of the sequence specific polynucleotide labeling process of the invention. The covalent binding step solves the problem of the specific DNA ligands losing their DNA interactions upon structural changes in the DNA

FIG. 2 is a stepwise description of the method of the invention.

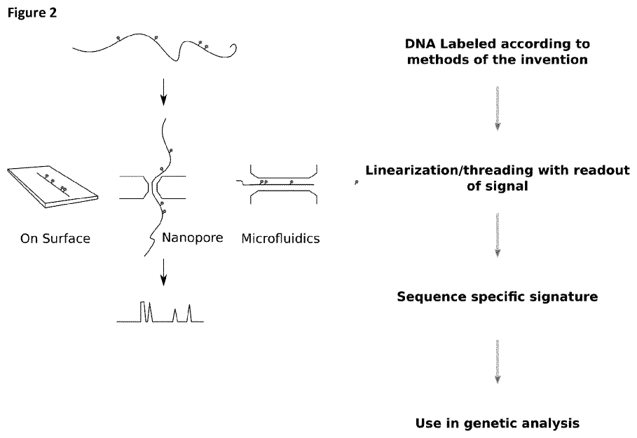

FIG. 3 is a schematic depiction of a specific embodiment of the invention where a signal is introduced after covalent binding of a sequence specific reagent

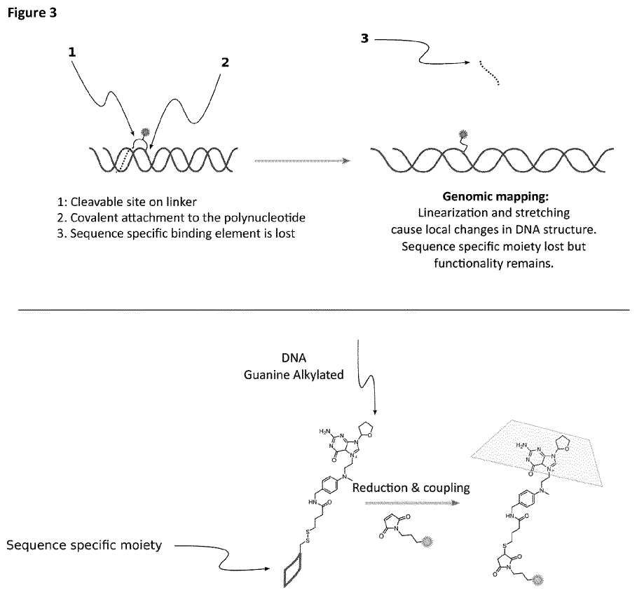

FIG. 4 are example sequence specific signatures of chemical agents capable of effecting the methods described in accordance with the claims

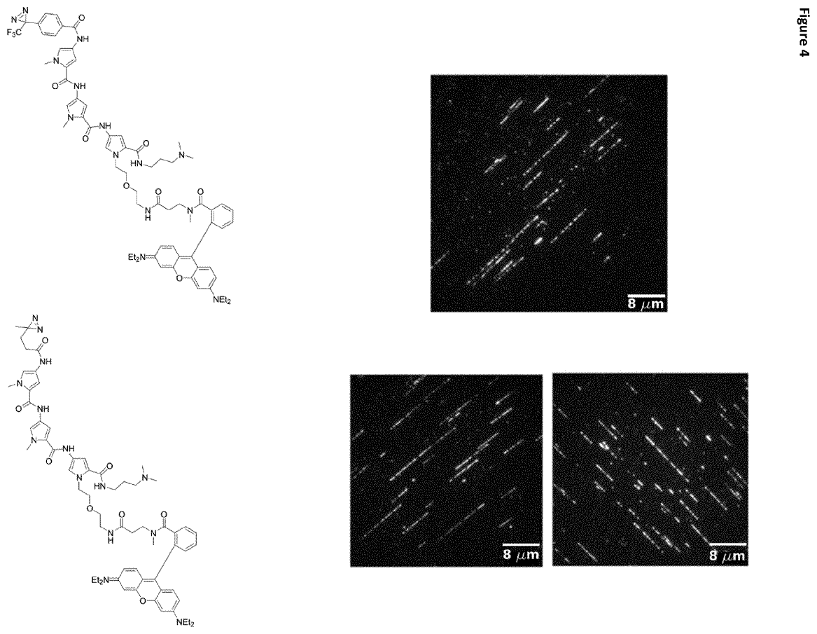

FIG. 5 is an example of the analysis of sequence specific signatures generated on a polynucleotide, as observed in fluorescence microscopy. Genomic maps of example 5 are assigned to the correct phage.

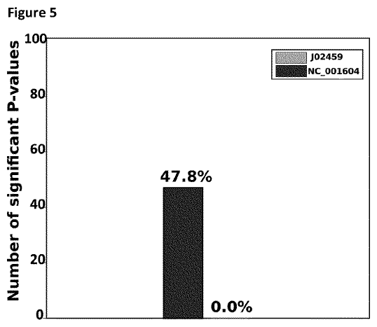

FIG. 6 is an example of a correct attribution of a genetic signature to an source of origin under competing conditions, indicating how covalent binding can maintain a sequence specific signature. Genomic maps of example 5 are assigned to the correct phage, despite stringent conditions.

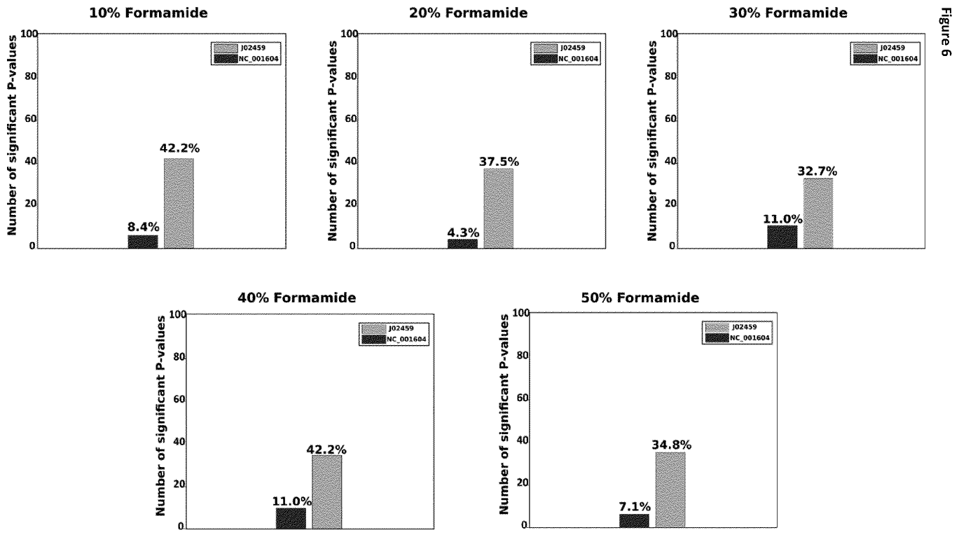

FIG. 7 shows the leeching of a non-covalently bound groove binder (Targeting AT rich regions) on double stranded DNA. At the moment of deposition (T=0) signal is observed over the entire DNA backbone. After 1 minute, the signal has already been largely lost due to leeching of the sequence specific groove binder from the backbone.

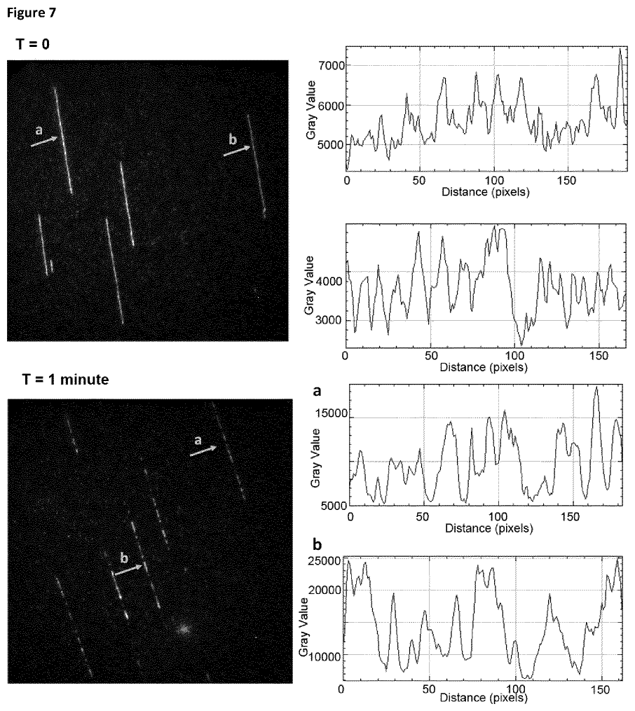

### DEFINITIONS

The following terms and related definitions are used in the present text.

“Subject” is used herein to mean any living being, human or animal. Nevertheless, the here disclosed method can be used for plants as well. As it is obvious for those skilled in the art, that subject in the context of this patent should mean any living body exposed to a viral infection.

“Sample” is used herein to mean first, any substance taken from a subject and undergoing a diagnosis based on the disclosed method. Secondly, our method applies equally well to any material like textiles, plastics, air filters, but not limited hereto. In summary, sample is used here for designating any living material and any solid or liquid or gaseous material where polynucleotides may be present. A sample taken from a subject may contain biological material such as saliva, mucus, cheek swabs, nasal swabs, blood, fecal matter, urine, or substances from breather masks, dust recovered from air filters, surface swabs but not limited hereto. For efficient early detection in populations these samples may be pooled

“Stretching” is used herein to mean depositing a DNA molecule onto a surface so that all vectors that point form a nucleotide n to the neighboring nucleotide n+1 or n−1 have a positive projection onto the vector from the first nucleotide to the last one. By these kind of approach the base pair distance is increased and acts like an additional magnification forl reading. Effectively this means that a DNA forms a linear object for at least a portion of its full length, where the DNA strand along the stretching may have up to several micrometer, but in the lateral, perpendicular to the stretching direction is limited to several nanometers.

“Optical read out” is used herein to mean: a method that uses light signals to glean a specific information allowing the identification with high accuracy of viral species. Such signal or optical intensity profiles are put into relation with the genetic codes known and downloaded from a databank. A matching algorithm, as for example based on a cross-correlation or a neuronal network, but not limited hereto serves to relate with high accuracy the measured signal to an priori known RNA or DNA based information, allowing to assign the measured signal to a known genetic information.

The term “substituted” as used herein refers to an organic group as defined herein or molecule in which one or more bonds to a hydrogen atom contained therein are replaced by one or more bonds to a non-hydrogen atom. The term “functional group” or “substituent” as used herein refers to a group that can be or is substituted onto a molecule, or onto an organic group. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents J that can be bonded to a substituted carbon (or other) atom include F, Cl, Br, I, OR′, OC(O)N(R′)2, CN, NO, NO2, ONO2, azido, CF3, OCF3, R′, O (oxo), S (thiono), C(O), S(O), methylenedioxy, ethylenedioxy, N(R′)2, SR′, SOR′, SO2R′, SO2N(R′)2, SO3R′, C(O)R′, C(O)C(O)R′, C(O)CH2C(O)R′, C(S)R′, C(O)OR′, OC(O)R′, C(O)N(R)2, OC(O)N(R′)2, C(S)N(R′)2, (CH2)O-2N(R′)C(O)R′, (CH2)O-2N(R′)N(R′)2, N(R′)N(R′)C(O)R′, N(R′)N(R′)C(O)OR′, N(R′)N(R′)CON(R)2, N(R′)SO2R′, N(R′)SO2N(R′)2, N(R′)C(O)OR′, N(R′)C(O)R′, N(R′)C(S)R′, N(R′)C(O)N(R′)2, N(R′)C(S)N(R′)2, N(COR′)COR′, N(OR′)R′, C(═NH)N(R′)2, C(O)N(OR′)R′, or C(═NOR′)R′ wherein R′ can be hydrogen or a carbon-based moiety, and wherein the carbon-based moiety can itself be further substituted; for example, wherein R′ can be hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or R′ can be independently mono- or multi-substituted with J; or wherein two R′ groups bonded to a nitrogen atom or to adjacent nitrogen atoms can together with the nitrogen atom or atoms form a heterocyclyl, which can be mono- or independently multi-substituted with J.

“Bioorthogonal” is used herein to mean: chemical reactions that can be used in biological systems, coupling one reactive group specifically with another reactive group: without side reactions; in neutral, aqueous solution; and under additional conditions that are compatible with the biological system. (Bioorthogonal Chemistry: Fishing for Selectivity in a Sea of Functionality, Ellen M. Sletten, Carolyn R. Bertozzi, Angew. Chem, 2009; The Future of Bioorthogonal Chemistry, Neal Devaraj, ACS Cent Sci, 2018, 4(8):95)

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference. Importantly, in this invention, the term “complementary” extends to the hybridization or pairing with a sequence specific agent that interacts with the nucleobases of the polynucleotide in a similar manner, through the formation of complementary hydrogen bonding patters. The nucleobases of a DNA are available for such hydrogen bonding in the grooves of the DNA, and therefore complementary groove binders can exist.

“Sequence specific” as used herein refers to binding of complementary nature to specific genetic elements. These genetic elements, or “specific sequences” can be sequences of nucelobases usually ranging from 2 to 20 basepairs, but preferentially 2-10 basepairs. Additionally, the specificity of the sequence binding is to include groups of similar genetic elements, or densities of genetic elemants, where hydrogen bonding patterns are similar. Such similar binding patterns can be readily deduced from footprinting experiments, pairing rules or spatial binding considerations.

“Nucleic acids” or “polynucleotides” of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or hybrids or combinations thereof.

By the phrase “nucleic acid extraction reagent” is meant any reagent (e.g., solution) that can be used to obtain a nucleic acid (e.g., DNA) from biological materials such as cells, tissues, bodily fluids, microorganisms, etc. An extraction reagent can be, for example, a solution containing one or more of: a detergent to disrupt cell and nuclear membranes, a proteolytic enzyme(s) to degrade proteins, an agent to inhibit nuclease activity, a buffering compound to maintain neutral pH, and chaotropic salts to facilitate disaggregation of molecular complexes.

“Reactive group” refers to a chemical moiety capable of reacting with a partner chemical moiety to for a covalent linkage. A moiety may be considered a reactive group based on its high reactivity with a single partner-moiety, a set of partner-moieties, or based on its reactivity with many partners.

“DNA Mapping” refers to a process where sequence specific markers are introduced to a polynucleotide, and where the distance information between these markers or the order in which different markers are present yields information on the genetic makeup of the polynucleotide. DNA mapping may refer to all polynucleotides in a sample, including but not limited to genomic DNA, plasmid DNA, mRNA, tRNA and genomic RNA.

### DETAILED DESCRIPTION OF INVENTION

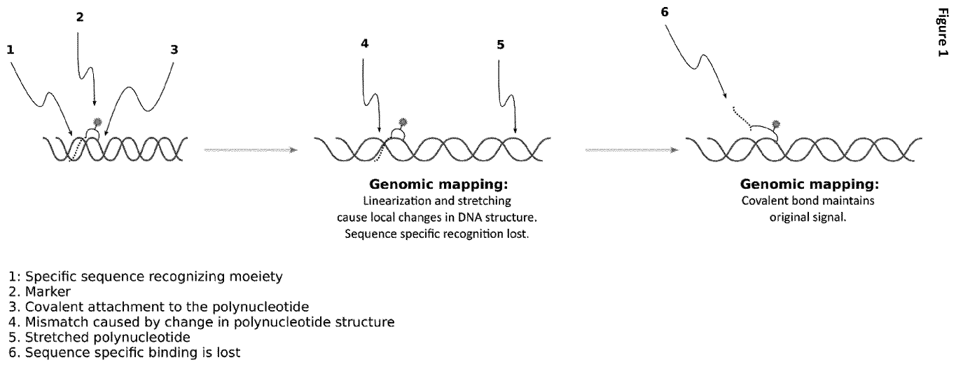

The disclosed method 100 is visualized in FIG. 1 and comprises 3 distinct steps, [10, 20,30], which can be subdivided as
A. Subjecting a polynucleotide to a covalent sequence specific labeling,
B. Linearizing said sequence specific labeled polynucleotide, and
C. Obtaining positional information on the sequence specific labels

In some embodiments, a method of covalently labeling a polynucleotide molecule at a target sequence is described (such methods may also be described herein as “labeling methods”). Thus, the polynucleotide can be covalently labeled by the labeling method. In some embodiments, the labeling of the method is performed in a single step.

In one embodiment, the method includes contacting DNA with a specific labeling agent comprising a portion, e.g. a binding sequence, complementary to the target sequence in the DNA, and configured to bind a label on the DNA at a specific location within, adjacent or near to the target sequence.

In some embodiments, the method further comprises detecting a relative distance between the labels on the linearized DNA, thus providing a barcode of a portion of the genomic DNA. In some embodiments, this distance can be detected by linearizing the labeled DNA in a fluidic channel, in which the DNA remains intact upon said linearization. In some embodiments, the distance can be detected by linearizing the labeled DNA on a surface. In some embodiments, the distance can be detected by passing the labeled DNA through a nanopore.

In some embodiments, the method is used for the analysis of polynucleotides. In some embodiments, the polynucleotide is genomic DNA. In some embodiments, the analysis of genomic DNA can be used for species identification, where these species are single species, or mixtures of species, as to identify the presence of species or the composition of the mixture of species.

In another embodiment, the genomic DNA is contacted with multiple sequence specific labeling agents, each agent having a portion complementary to a different target sequence in the genomic DNA, but not necessarily with different labels, and wherein each target nucleic acid sequence is detected via the same or different label, thus providing a barcode of a portion of the genomic DNA. In some embodiments, the method further comprises labeling the DNA by an additional chemistry, for example direct enzymatic labeling using an enzyme and optionally further including a stain in addition to the enzymatic labeling, or nicking followed by nick labeling and repair to produce a DNA with two or more different specificity motifs with different labels (e.g., different colors).

In the labeling methods, DNA compositions, and kits of some embodiments disclosed herein, a non-enzymatic sequence specific DNA ligand is used to label selected target sequences on DNA.

According to the labeling methods, and kits of some embodiments herein, a polynucleotide is labeled using sequence specific polynucleotide ligands that form a covalent bond with the polynucleotide. Advantageously, the sequence specific DNA ligand stably binds its target, providing a sequence specific label on the genomic DNA at a specific location within or adjacent to the target sequence. When multiple labels are introduced onto the DNA, the relative distance between the labels on the DNA can be measured. This distance information can then provide insights into DNA structure and identity. Since the target sequences of these ligands can be tuned at will, this provides a solution to the limitations in available target sequences observed with enzymatic DNA labeling approaches. When multiple labels are introduced, the absolute or relative amount of each of the labels is a measure of the presence of certain genetic elements on the DNA, and therefore also a identifier of said DNA. A non-specific DNA stain can also be used to provide a measure of DNA length at the same time.

The ligand or sequence specific labeling agents as used herein, contain a reactive group which can react covalently with the DNA within or adjacent to the target sequence. Advantageously, such covalent attachment of the label ensures retention of the label within or adjacent to the target sequence during changes in the DNA structure, conformation and DNA helix pitch as are routinely observed in genomic mapping processes. The methods of the invention thus provide a solution for using non-enzymatic sequence specific DNA labeling enabling unprecedent approaches in polynucleotide mapping.

Additionally, some embodiments of the invention allow the covalent labeling of polynucleotides at or near a site of specific binding of a sequence specific ligand, followed by cleavage of any linker or bond existing between the covalently bound label and the sequence specific ligand. The sequence specific ligand remains in such a case only bound to the polyncuelotide by non-covalent bonds, and may dissociate from the polynucleotide. It may be advantageously to effect this dissociation from the polynucleotide, since the sequence specific ligand and its polynucleotide interactions provide local rigidification or condensation (Nyberg et al Biochem Biophys Res Commun. 2012 Jan 6;417(1):404) and will lead to local differences in linearization length between labels. When dissociated, the polynucleotide can linearize or stretch more uniformly over its total length, leading to improved analysis of the sequence specific labeling patterns.

In some embodiments, the labeled polynucleotide has a length in the kilobase or megabase range, for example at least 1 kb, 2 kb, 3kb, 5 kb, 10 kb, 20 kb, 30 kb, 40 kb, 50 kb, 100 kb, 150 kb, 250 kb, 500 kb, 1 Mb, 1.5 Mb, or 2 Mb, including ranges between any two of the listed values (for example 1 kb-2 Mb, 5 kb-2 Mb, 10 kb-2 Mb, 20 kb-2 Mb, 100 kb-2 Mb, 500 kb-2 Mb, 1 kb-1 Mb, 5 kb-1 Mb, 10 kb-1 Mb, 100 kb-1 Mb, 200 kb-1 Mb, 500 kb-1 Mb, 1 kb-500 kb, 5 kb-500 kb, 10 kb-500 kb, 20 kb-500 kb, 100 kb-500 kb, 1 kb-100 kb, 5 kb-100 kb, 10 kb-100 kb, 20 kb-100 kb, 50 kb-100 kb, 1 kb-50 kb, 5 kb-50 kb, 10 kb-50 kb, 1 kb-10 kb, 5 kb-10 kb, or 1 kb-5 kb).

In some embodiments, the covalently labeling method includes covalently labeling the polynucleotide at two or more different target sequences using different labels for each target sequence. Accordingly, the labeling method or complex of some embodiments, further comprises two or more sequence specific labels that each comprises a sequence specific ligand that is complementary to a different target sequences or portion(s) thereof of the polynucleotide, so that different target sequences on the polynucleotide are labeled with different labels. In some embodiments, each target sequence is labeled with a unique label. For example, the labeling method can comprise contacting the polynucleotide with a first sequence specific ligand comprising a first label complementary to a first target sequence (or portion thereof) on the polynucleotide, a second sequence specific ligand comprising a second label that is different from the first target sequence and complementary to a second target sequence (or portion thereof) on the polynucleotide that is different from the first target sequence, and/or a third sequence specific ligand comprising a third label that is different from the first label and/or the second label and complementary to a third target sequence (or portion thereof) on the polynucleotide that is different from the first target sequence and/or the second target sequence. In some embodiments, the polynucleotide is contacted with the different labels at the same time, for example in a single composition. In some embodiments, the polynucleotide is contacted with the different labels separately. (for example, if the first and second compositions are added sequentially). Advantageously, such multitarget and multilabel methods provide a solution to variations in signal sometimes observed with polynucleotide sections containing low number of target sequences.

In certain embodiments, these non-enzymatic sequence specific polynucleotide ligands comprise a portion, i.e. a sequence specific structure that recognizes specific sequence elements through specific interaction with patterns of nucleobases. These interactions can for example take place through direct hybridization with the polynucleotide chain or through interactions with structural elements of the polynucleotide molecules, such as the major and minor groove in DNA molecules. Example of such specific binding portions in the non-enzymatic sequence specific polynucleotide ligands according to the invention can be selected from the range of but not limited to benzimidazole dimers and oligomers, pyrrole oligomers, flavones, pyrrole-imidazole oligoamides, synthetic oligodeoxynucleotides (ODN), triple-helix forming oligonucleotides, or a combination of two or more of the listed items.

In certain embodiments, cationic DNA ligands exhibit a sequence specificity, with such examples as Hoechst 33342, Hoechst 33258 and 34580 displaying preference for AT rich sequences. Synthetic alternatives allow for tuning of the specificity. Further examples of such sequence specific structures are described in J. Gonzalez-Garcia, et al. (2017) “Supramolecular Principles for Small Molecule Binding to DNA Structures”, 39-70 and Nelson S. M., et al. (2007), “Non-covalent ligand/DNA interactions: Minor groove binding agents Mutation Research/Fundamental and Molecular Mechanisms of Mutagenesis”, 623, 24-40, each of which is hereby incorporated by reference in its entirety.

In certain embodiments, polypyrrole ligands and related lexitropsin structures exhibit sequence specificity. Importantly, synthetic alternatives allow for tuning of the sequence specificity. These structures can be further elaborated in polyamides consisting of sequences of heterocycles, where the sequence of heterocyclic rings allows for tuning of the target sequence. Examples of such sequence specific structures are described by Dervan (Curr Opin Struct Biol., 2003, 284-99), hereby incorporated by reference in its entirety.

A notable and previously undescribed advantage of using such heterocyclic oligomers is their capacity to bind multiple times at the same location Thus, two labels are introduced at or near a single site of the DNA, leading to increased signal-over-noise.

In certain embodiments, synthetic oligodeoxynucleotides (ODN) have shown the capacity to bind to double-stranded DNA and form a so-called triple-helix synthetic oligodeoxynucleotide The ODN winds around the DNA in the major groove and binding is stabilized through the formation of Hoogsteen-type hydrogen bonds. These triple-helix forming ODNs will preferentially bind to homopurine/homopyrimidine sequences. Often, an additional stabilization of the triple-helix is achieved by covalently linking the overhanging end using DNA ligases or through the activation of a photo-reactive group present on the synthetic oligodeoxynucleotide.

In certain embodiments, flavones exhibit a sequence specificity, with such examples as Kanwal R., (2016) “Dietary Flavones as Dual Inhibitors of DNA Methyltransferases and Histone Methyltransferases” PLoS One. 2016; 11(9): e0162956., displaying preference for GC rich sequences.

In certain embodiments, direct hybridization of oligonucleotides (DNA, LNA, CNA, PNA) occurs. In certain embodiments, this is brought to effect through either direct hybridization with partial melting or through triple helix formation. Examples of such sequence specific structures are described Gottfried A. et al. “Sequence-specific covalent labelling of DNA”, Biochemical Society Transation, 39(2), 623-628, hereby incorporated by reference in its entirety

The principles here described can be extended to the specific labeling and analysis of RNA, through the use of sequence specific RNA ligands. Examples of such ligands are described in Aboul Ela, (2010) “Strategies for the design of RNA-binding small molecules” Future Medicinal Chemistry,. 2(1)

In addition to the sequence specific structure, the sequence specific DNA ligands according to the invention further comprises a reactive moiety that allows covalent placement of the label on the genomic DNA at a location within or adjacent to the target sequence. Thus far all attempts to expand the existing DNA labeling methods into DNA mapping on surfaces or on overstretched DNA have failed, as the DNA manipulation changes the actual physical properties of the DNA that allow for sequence recognition. For example, we found that DNA stretching on surface with fluorescent sequence specific groove binding agents is not able to generate a sequence specific signature in DNA mapping, as the changing pitch of the DNA upon linearization also changes the binding characteristics and hydrogen bonding patterns of the thus connected DNA labels. These effects are strengthened further when the DNA is stretched beyond its solution phase length, or overstretched. The changing binding characteristics cause the DNA binding agents to change or loose its DNA specificity and binding strength. As such, sequence specific information is not retained. Advantageously, the proposed methods of covalent labeling are able to overcome the aforementioned physical changes, with retention of genomic information signature. Advantageously, the methods described also reduce the impact of other solution components, such as salts or DNA stabilizing or destabilizing agents, often encountered in buffers for linearization, which cause reduced specificity or leeching of the sequence specific agent.

The reactive moiety will form a covalent bond with the polynucleotide. This covalent bond can be formed with all components of the polynucleotide chain, such as ribose chain elements, phosphate chain elements or nucleobases. Reactive groups capable of doing so are, amongst others, platinum complexes, electrophiles (such as mustards, aziridines), nitrenes, carbenes and ng. The labeling may be initiated at a time of choosing, through for example heating or light, and the reactive moiety may be generated from a precursor, such as a nitrene from an azide.

In certain embodiments, the sequence specific DNA ligands will comprise a label. In some embodiments, the labels can be, for example, a fluorophore, a quantum dot, a dendrimer, a nanowire, a bead, a hapten, a streptavidin, an avidin, a neutravidin, a biotin, a reactive group, a peptide, a protein, a magnetic bead, a radiolabel, a non-optical label, or a combination of two or more of the listed items.

By combining the sequence specific DNA ligand with a label, and producing a complex that targets a genomic region, labeling can be accomplished by direct binding.

In certain embodiments, the label can be cleaved from the sequence specific DNA ligand after covalent attachment of the sequence specific DNA ligand to the polynucleotide. This detachment of label, can for example be triggered by enzymes, nucleophiles, electrophiles, shifts in pH, oxidation and oxidative or reductive cleavage of chemical bonds.

In certain embodiments, the sequence specific DNA ligand carries reactive groups which can react with labels after covalent attachment to the polynucleotide. These reactive groups are preferably bioorthogonal in reactivity.

In some embodiments, the labeling method, further comprises labeling the DNA by an additional chemistry, for example direct enzymatic labeling using a methyltransferase enzyme and optionally further including a stain in addition to the enzymatic labeling, or nicking followed by nick labeling and repair to produce a DNA with two or more specificity motifs (such as target sequences) labeled with different labels (e.g., different colors). In some embodiments, the nick labeling comprises nicking the DNA with a modified restriction enzyme which cuts a single strand (nickase) instead of both strands. Labeled nucleotides can then be incorporated into the nicked DNA directly (optionally, followed by repair), or by nick translation. Optionally, the DNA can be repaired with ligase following the nick translation. Optionally, the DNA can also be stained with a non-specific backbone label, such as a YOYO label. The nonspecific label can be added after the sequence specific labeling, or can be present during the sequence specific labeling.

In some embodiments, the labeling method, in addition to labeling with sequence specific ligand, further comprises labeling the DNA by an additional chemistry, for example direct enzymatic labeling using an enzyme and optionally further including a stain in addition to the enzymatic labeling to produce a DNA comprising two or more specificity motifs (such as target sequences) with different labels (e.g., different colors). It is contemplated that labeling multiple specificity motifs with multiple colors can yield greater information density than labeling a fewer number of motifs. Advantageously, the labeling methods herein can be accomplished with a simple protocol that only requires incubation, and it is non damaging to DNA. This damaging of DNA can cause double-stranded breakage of damaged DNA, confounding the analysis of labeling patterns. Without being limited by theory, it is contemplated the labeling methods herein can achieve labeling more rapidly, and be used to target a greater variety of target sequences than enzymatic DNA labeling.

Sequence-specific labeling in accordance with the methods and kits of some embodiments described herein can be useful in genomic mapping. This single-step labeling of some embodiments does not damage the polynucleotide, and the flexible and efficient tagging of specific sequences enables acquisition of context-specific sequence information, when performing single-molecule mapping of polynucnleotide. Not only can the methods and kits of some embodiments yield superior quality and sensitivity of whole-genome structural variation analysis by adding a second color and increasing information density, it is also able to target a wide variety of sequences such as long tandem repeats, viral integration sites, transgenes, and can even be used to genotype single nucleotide variants.

Methods of labeling polynucleotides described herein can be useful in, for example, identification of species, analysis of mixtures of species, analysis of biomes. In some embodiments, the method can be used for the analysis of genomic DNA, targeting repetitive sequences, barcoding genomic regions and structural variants not amenable to enzymatic motif-based labeling, where uneven distributions of the targeted sequence motifs in the DNA can lead to inaccurate assignment. This rapid, convenient, non-damaging and cost-effective technology provides a valuable tool for both automated high-throughput species identification and species mixture analysis, as well as genome-wide mapping, targeting complex regions containing repetitive and structurally variant DNA.

As certain specific DNA binding agents have been shown to be sensitive to epigenetic DNA modifications, (e.g. Minoshima, Nucleic Acids Research, Volume 36, Issue 9, 1 May 2008, Pages 2889-289), it is contemplated that the reagents can have use the analysis of epigenetic status of polynucleotides and their application.

It is contemplated that in the labeling methods, DNA compositions, and kits of some embodiments, two or more different target sequences of a DNA can be labeled. Accordingly, it is contemplated that in the labeling methods, DNA compositions and kits of some embodiments, the two or more target sequences can have a different label. Accordingly, in the labeling methods, DNA compositions and kits of some embodiments, the DNA labeling is multiplex.

The methods described are to be combined with polynucleotide linearization, where molecular combing is one exemplary method for stretching and immobilizing DNA. Molecular combing is a highly parallel process that can produce high-density packed long DNA molecules stretched on a surface. The DNA strands can range in size from several hundred Kb to more than 1 Mb. In one embodiment, molecular combing is a process through which free DNA in a solution can be placed in a reservoir, and a hydrophobic-coated slide is dipped into the DNA solution and retracted. Retracting the slide pulls the DNA in a linear fashion. Functionalized slides and combing devices based on this approach are currently commercially available. Alternatively, DNA linearization can be achieved by other methods, where a receding meniscus drags and stretches DNA on a surface (Deen et al, ACS Nano).

Fluidic channels can be useful for the analysis of structural features of linearized DNA, both for long (e.g., kilobase, or megabase-length) DNA molecules as well as short DNA molecules. Detailed information on suitable fluidic channels can be found, for example, in U.S. Pat. Nos. 8,722,327, 8,628,919, and 9,533,879, each of which is hereby incorporated by reference in its entirety. Suitable channels for the labeling methods, DNA compositions, and kits of some embodiments, can have, for example, a diameter of less than about twice the radius of gyration of the macromolecule in its extended form. A nanochannel of such can exert entropic confinement of the freely extended, fluctuating DNA coils so as to extend and elongate the DNA.

Accordingly, in the labeling methods, DNA compositions, and kits of some embodiments, the fluidic nanochannel is capable of linearizing the DNA molecule (so as to entropic confinement of the DNA coils so as to extend and elongate the DNA molecule). Upon linearization in a fluidic nanochannel, the DNA molecule is maintained in a linearized, stretched conformation that permits the determination of the relative positions of labels along the length of the DNA. Such labels can be used to assign origin of the DNA within a larger DNA, study DNA structural variations such as complex rearrangements, haplotype analysis, quantification of copy number of repeater elements on long (kilobase or megabase-scale) DNA, quantify short DNAs, resolve multiple repeats, insertions, and/or to assemble sequences or labeling patterns indicative of DNA structures onto a scaffold.

In some specific embodiments, the labeled polynucleotide can be translocated through a nanonopore. In such a case, the sequence specific signal can be observed through for example electrical or optical methods. Noteworthy, the linearization of the polynucleotide is only local in such a case, at and near the portion of the polynucleotide transferring through the pore. O Combining the information of the entire polynucleotide as it passes through the pore allows to reconstruct The distance information into a sequence specific signature over the entire polynucleotide. The signal can be observed as a change in voltage or current as a label on the polynucleotide passes through the pore.

In some embodiments, the method further comprises labeling the DNA by an additional chemistry, for example direct enzymatic labeling using a methyltransferase enzyme or nicking enzyme followed by incubating the nicked DNA with a polymerase and labeled nucleotides

As disclosed herein, non-limiting exemplary labels include: a fluorophore, a quantum dot, a dendrimer, a nanowire, a bead, a hapten, a streptavidin, an avidin, a neutravidin, a biotin, a reactive group a peptide, a protein, a magnetic bead, a radiolabel, a non-optical label, and a combination of two or more of the listed items. In some embodiments, the label is an optical label. If the labeling method comprises two or more different labels, then two or more of the labels can be of the same types (for example two different fluorophores), or two or more of the different labels can be of two or more different types (for example, a fluorophore and a quantum dot), or a combination of two or more of the listed items.

Exemplary labels are well known in the art (see, for example, U.S. Pat. No. 6,323,337; WO 00/58505 (PCT/EP99/07127) and references cited therein, Hermanson, Bioconjugate Techniques, Academic Press, San Diego (1996), each of which is incorporated herein by reference).

In some embodiments, the DNA is further labeled with a nonspecific label, for example a backbone label, such as YOYO-1 label (the nonspecific label may also be referred to herein as a “stain”). Other examples of such stains include but are not limited to DAPI, POPO-1, BOBO-1, JOJO-1, POPO-3, LOLO-1, BOBO-3, YOYO-3, TOTO-3, Ethidium Bromide, SYBR-SAFE. The nonspecific label can be added after the sequence specific labeling. In some embodiments, the sequence specific and nonspecific labeling of the method are performed in a single step.

Some embodiments include a kit for performing any of the labeling methods described herein. The kit can comprise a sequence specific agent as described herein. The kit can comprise multiple sequence specific agents In some embodiments, the kit further comprises a label. In some embodiments, the label is not attached to the sequence specific agent. In some embodiments, the kit further comprises In some embodiments, the kit further comprises a nickase. In some embodiments, the kit further comprises a direct labeling enzyme such as a methyltransferase.

The method is rapid, convenient, cost-effective, and non-damaging. The flexible and efficient fluorescent tagging of specific sequences allows the ability to obtain context specific sequence information along the long linear DNA molecules in DNA mapping. Not only can this integrated fluorescent DNA double strand labeling make the whole genome mapping more accurate, and provide more information, but it can also specifically target certain loci for clinical testing, including detection of SNPs. Additionally, it can render the labeled double-stranded DNA available in long intact stretches for high-throughput analysis in nanochannel arrays as well as for lower throughput targeted analysis of labeled DNA regions using alternative methods for stretching and imaging the labeled large DNA molecules. Thus, labeling methods of some embodiments dramatically improve both automated high-throughput genome-wide mapping as well as targeted analyses of complex regions containing repetitive and structurally variant DNA. Thus, the method and some embodiments herein allow for developing combinatorial, multiplexed, multicolor imaging systems, and thus can offer advantages for rapid genetic diagnosis of structural variations.

### EXAMPLES

### Example 1

Non-exhaustive list of examples of reagents used in the invention with some of their traits.

Reagent 1: Pyrrole-Imidazole oligomer with appending green fluorescent dye and arylazide for
covalent binding to polynucleotide

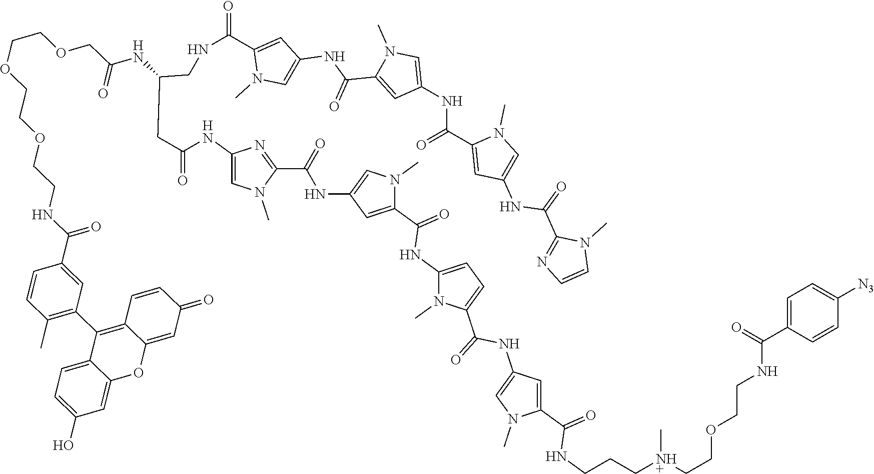 [SMILES: Cc1ccc(C(=O)NCCOCCOCCOCC(=O)N[C@H](CNC(=O)c2cc(NC(=O)c3cc(NC(=O)c4cc(NC(=O)c5nccn5C)cn4C)cn3C)cn2C)CC(=O)Nc2cn(C)c(C(=O)Nc3cc(C(=O)Nc4ccc(C(=O)Nc5cc(C(=O)NCCC[NH+](C)CCOCCNC(=O)c6ccc(N=[N+]=[N-])cc6)n(C)c5)n4C)n(C)c3)n2)cc1-c1c2ccc(=O)cc-2oc2cc(O)ccc12]

Reagent 2: Bis-benzimide DNA binder with appended nitrogen mustard and Rhodamine 6G dye
for AT-rich region targeting

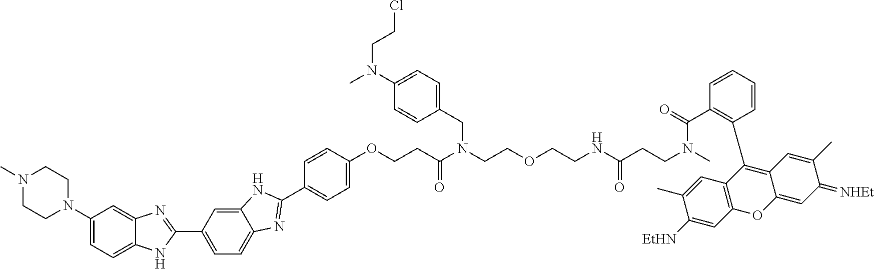

Reagent 3: Distamycin analog for AT-rich region targeting, comprising a nitrogen mustard for
covalent binding, a Rhodamine B dye and a cleavable linker to release the sequence specific
moiety.

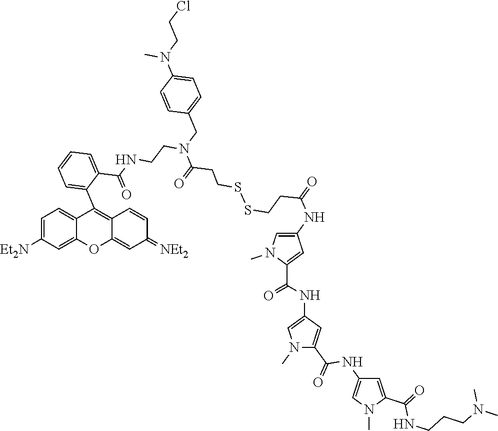

Reagent 4: Netropsin analog for AT-rich region targeting, comprising a diazirine for thermal or
photoactivatable covalent binding and an aliphatic azide for biorthogonal labeling or capture.

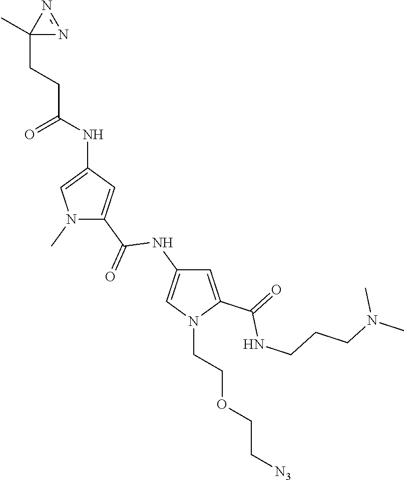

Reagent 5: Netropsin analog for AT-rich region targeting, comprising a diazirine for thermal or
photoactivatable covalent binding and a Rhodamine B dye for direct visualization of the genetic
signature.

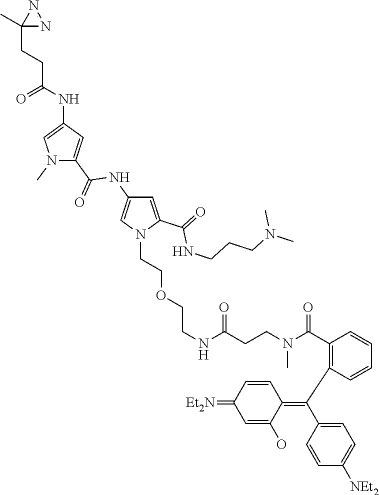

Reagent 6: Heterocycle oligomer for GTAA targeting, comprising an alkylating
duocarmycincovalent binding and and an aliphatic azide for biorthogonal labeling or capture.

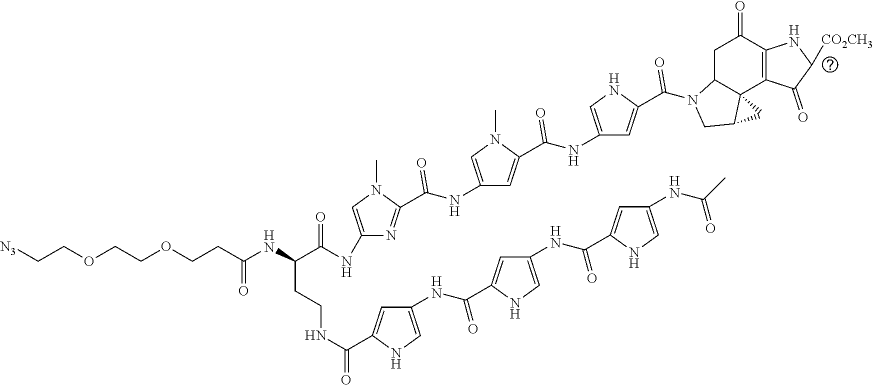 [SMILES: COC(=O)C1NC2=C(C1=O)[C@@]13C[C@@H]1CN(C(=O)c1cc(NC(=O)c4cc(NC(=O)c5nc(NC(=O)[C@@H](CCNC(=O)c6cc(NC(=O)c7cc(NC(=O)c8cc(NC(C)=O)c[nH]8)c[nH]7)c[nH]6)NC(=O)CCOCCOCCN=[N+]=[N-])cn5C)cn4C)c[nH]1)C3CC2=O]

Reagent 7: Distamycin analog for targeting AT-rich DNA-sequences and tetrades of [TGGGGT]₄
comprising nitrogen mustard for covalent binding and a cleavable linker which generates a
reactive thiol upon cleavage, for further reaction with e.g. a maleimide containing dye.

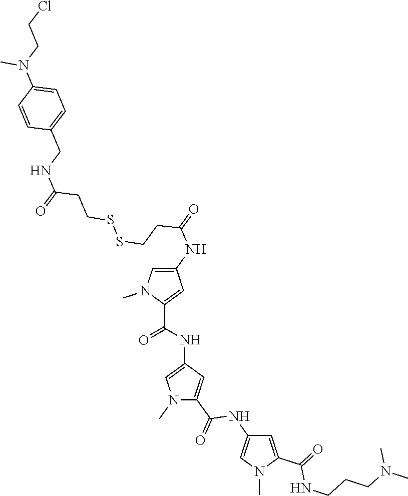

Reagent 8: Lexitropsin analog for GC-rich region targeting, comprising a platinum complex for
covalent binding and a cleavable linker which generates a reactive thiol upon cleavage, for
further reaction with e.g. a maleimide containing dye.

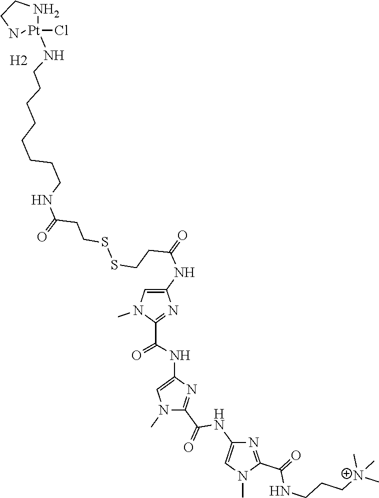

Reagent 9: Double linked pyrrole-imidazole oligomer for WGWWCW (W = A or T), including a
double nitrogen mustard for covalent binding to DNA and an azide moiety for reaction with e.g.
alkyne containing labels.

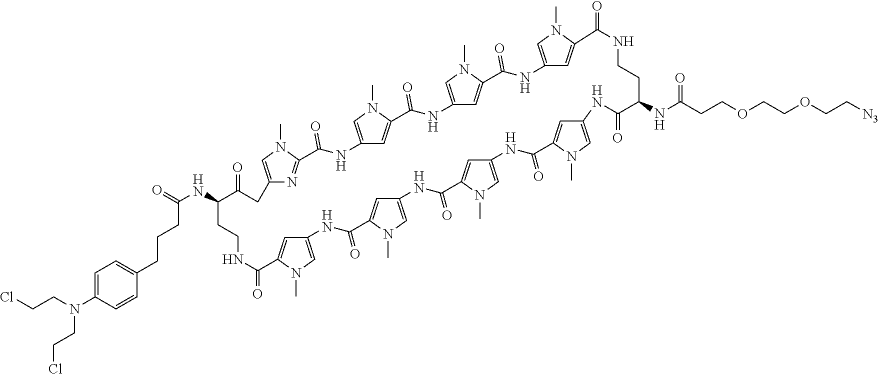 [SMILES: Cn1cc2cc1C(=O)NCC[C@@H](NC(=O)CCCc1ccc(N(CCCl)CCCl)cc1)C(=O)Cc1cn(C)c(n1)C(=O)Nc1cc(n(C)c1)C(=O)Nc1cc(n(C)c1)C(=O)Nc1cc(n(C)c1)C(=O)NCC[C@@H](NC(=O)CCOCCOCCN=[N+]=[N-])C(=O)Nc1cc(n(C)c1)C(=O)Nc1cc(n(C)c1)C(=O)Nc1cc(n(C)c1)C(=O)N2]

Reagent 10: Distamycin analog AT-rich DNA-sequences and tetrades of [TGGGGT]₄ comprising a
diazirine for covalent binding and a Rhodamine B dye for direct imaging.

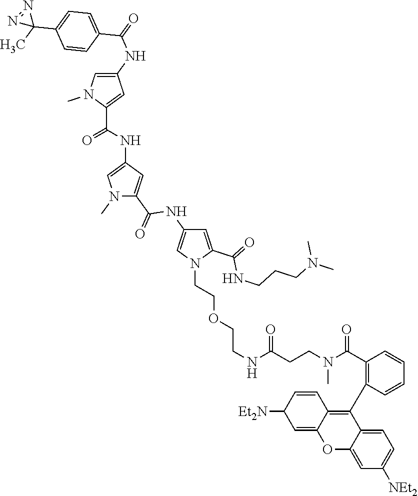

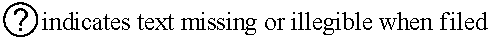

### Example 2

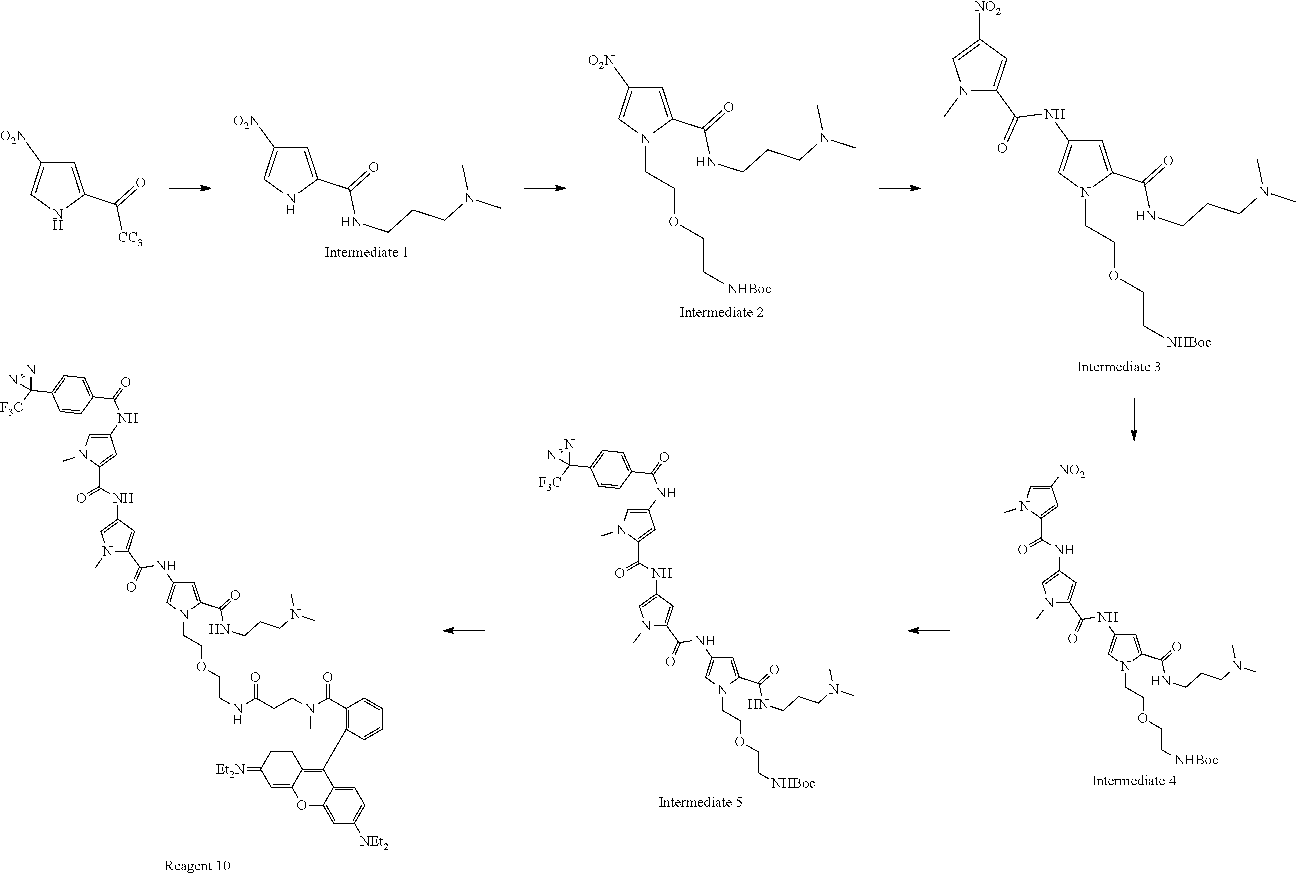

Reagent 10 was prepared in line with literature procedures and according to the scheme above. In brief, Nitro trichloroacetylpyrroles (6.89 g, 26.76 mmol) was dissolved in 1,4-dioxane (108 mL). At rt, 3-(dimethylamino)-1-propylamine (3.54 mL, 2.8712 g, 28.10 mmol, 1.05 equiv.) was added and the reaction was stirred for 30 min. After completion, the precipitate was filtered off, washed with cold dioxane and pentane and dried on high vacuum. Intermediate 1 was obtained as a white solid (5.23 g) in 81% yield. ¹H NMR (300 MHz, DMSO) δ 12.65 (br s, 1H), 8.41 (t, J=4.8 Hz, 1H), 7.89 (s, 1H), 7.41 (s, 1H), 3.24 (dd, J=12.6, 6.4 Hz, 2H), 2.24 (t, J=7.0 Hz, 2H), 2.13 (s, 6H), 1.74-1.54 (m, 2H). ¹³C NMR (75 MHz, DMSO) δ 159.1, 136.1, 127.1, 122.1, 104.7, 56.5, 44.9, 37.0, 26.9. HRMS (ES+): calculated for C₁₀H₁₆N₄O₃ [M+H]+: 241.1295 Found: 241.1297.

Intermediate 1 (2.40 g, 10 mmol), 2-[2-(Boc-amino)ethoxy]ethanol (2.26 g, 11 mmol, 1.1 equiv.) and triphenylphosphine (2.89 g, 11 mmol, 1.1 equiv.) were dissolved in dry THF (50 mL) and the resulting suspension was cooled to 0° C. At 0° C., DEAD (2.2M in toluene, 5 mL, 11 mmol, 1.1 equiv.) was added dropwise and the reaction was stirred at rt overnight. After completion, the solvent was removed and intermediate 2 was obtained after purification by column chromatography (silica, DCM/MeOH, 85/15) as a yellow viscous oil (3.47 g) in 81% yield. ¹H NMR (300 MHz, CDCl₃) δ 8.75 (br s, 1H), 7.64 (s, 1H), 6.99 (s, 1H), 4.76 (s, 1H), 4.59 (t, J=4.7 Hz, 2H), 3.76 (t, J=4.8 Hz, 2H), 3.56-3.41 (m, 4H), 3.29-3.22 (m, 2H), 2.61-2.50 (m, 2H), 2.37 (s, 6H), 1.82-1.72 (m, 2H), 1.43 (s, 9H). ¹³C NMR (101 MHz, CDCl3) δ 160.4, 156.0, 135.2, 126.8, 126.3, 107.1, 79.6, 70.4, 70.2, 59.4, 50.0, 45.4, 40.3, 28.5, 24.7. HRMS (ES+): calculated for C₁₉H₃₃N₅O₆ [M+H]+: 428.2503 Found: 428.2504.

Intermediate compound 2 (2.02 g, 4.72 mmol) in MeOH (20 mL) was subjected to Pd/C (10%) under a hydrogen atmosphere for 3 hours, filtered and evaporated to dryness. N-Methyl-4-Nitro-2-carboxy pyrrole (0.669 g, 3.93 mmol) and HBTU (1.64 g, 4.33 mmol) were dissolved in dry DMF (10 mL). Triethylamine (1.65 mL, 11.80 mmol) was added and the reaction was stirred at rt for 1 hour. After completion, the crude amine in dry DMF (5 mL) was added and the whole was stirred at rt overnight. After completion, the mixture was poured in H₂O (150 mL) and extracted with EtOAc (3×150 mL). The combined organics were washed with brine, dried over Na₂SO₄, filtered and evaporated. Intermediate 3 was obtained after purification by column chromatography (silica, DCM/MeOH, 8/2) as a yellow foam (1.91 g) in 88% yield. ¹H NMR (300 MHz, DMSO) δ 10.24 (s, 1H), 8.22-8.10 (m, 2H), 7.58 (s, 1H), 7.26 (s, 1H), 6.84 (s, 1H), 6.76-6.64 (m, 1H), 4.47-4.36 (m, 2H), 3.96 (s, 3H), 3.67-3.54 (m, 2H), 3.33 (t, J=5.8 Hz, 2H), 3.22-3.16 (m, 2H), 3.07-2.98 (m, 2H), 2.31 (t, J=6.9 Hz, 2H), 2.19 (s, 6H), 1.69-1.54 (m, 2H), 1.36 (s, 9H). 13C NMR (101 MHz, DMSO) δ 161.1, 156.9, 155.6, 133.8, 128.2, 126.3, 122.8, 121.4, 117.6, 107.5, 104.4, 77.6, 70.4, 69.0, 56.9, 47.5, 45.0, 37.5, 37.0, 28.2, 27.0. HRMS (ES+): calculated for C₂₅H₃₉N₇O₇ [M+H]+: 550.2984 Found: 550.2983.

Intermediate 3 (1.91 g, 3.48 mmol) in was reduced under hydrogen atmosphere in the presence of Pd/C in MeOH (17.5 mL). Reaction time for hydrogenation was 4 hours, followed by filtration and evaporation to dryness. N-methyl-4-Nitro-2-carboxy pyrrole (0.494 g, 2.90 mmol) and HBTU (1.21 g, 3.19 mmol) were dissolved in dry DMF (10 mL). Triethylamine (1.21 mL, 8.71 mmol) was added and the reaction was stirred at rt for 1 hour. After completion, the crude amine in dry DMF (5 mL) was added and the whole was stirred at rt overnight. After completion, the mixture was poured in H₂O (150 mL) and extracted with EtOAc (3×150 mL). The combined organics were washed with brine, dried over Na₂SO₄, filtered and evaporated. Intermediate 4 was obtained after purification by column chromatography (silica, DCM/MeOH, 75/25) as a yellow foam (1.74 g) in 89% yield. ¹H NMR (600 MHz, DMSO) δ 10.30 (s, 1H), 9.94 (s, 1H), 8.19 (d, J=1.7 Hz, 1H), 8.11 (t, J=5.6 Hz, 1H), 7.61 (d, J=1.9 Hz, 1H), 7.28 (d, J=1.7 Hz, 1H), 7.25 (d, J=1.6 Hz, 1H), 7.04 (d, J=1.7 Hz, 1H), 6.86 (d, J=1.7 Hz, 1H), 6.72 (t, J=5.5 Hz, 1H), 4.40 (t, J=5.5 Hz, 2H), 3.97 (s, 3H), 3.86 (s, 3H), 3.60 (t, J=5.5 Hz, 2H), 3.34 (t, J=6.1 Hz, 2H), 3.19 (dd, J=12.7, 6.7 Hz, 2H), 3.04 (dd, J=11.8, 5.9 Hz, 2H), 2.30 (t, J=7.0 Hz, 2H), 2.18 (s, 6H), 1.63 (quintet, J=7.0 Hz, 2H), 1.36 (s, 9H). ¹³C NMR (151 MHz, DMSO) δ 61.2, 158.4, 156.9, 155.6, 133.8, 128.2, 126.3, 123.1, 122.5, 122.1, 121.4, 118.6, 117.5, 107.6, 104.6, 104.5, 77.6, 70.4, 69.0, 56.9, 47.4, 45.0, 37.5, 37.0, 36.2, 28.2, 27.0. HRMS (ES+): calculated for C₃₁H₄₅N₉O₈ [M+H]+: 672.3464 Found: 672.3480.

From intermediate compound 4 (0.503 g, 0.75 mmol) in MeOH (3.75 mL). Reaction time for hydrogenation was 3 hours. 4-[3-(Trifluoromethyl)-3H-diazirin-3-yl]benzoic acid (0.144 g, 0.62 mmol) and HBTU (0.260 g, 0.69 mmol) were dissolved in dry DMF (3 mL). Triethylamine (0.26 mL, 1.87 mmol) was added and the reaction was stirred at rt for 1 hour. After completion, the crude amine in dry DMF (0.5 mL) was added and the whole was stirred at rt overnight. After completion, the mixture was poured in H₂O (50 mL) and extracted with EtOAc (3×50 mL). The combined organics were washed with brine, dried over Na₂SO₄, filtered and evaporated. Intermediate compound 5 was obtained after purification by column chromatography (silica, DCM/MeOH, 65/35) as a yellow foam (0.197 g) in 37% yield. ¹H NMR (600 MHz, DMSO) δ 0.51 (s, 1H), 10.00 (s, 1H), 9.90 (s, 1H), 8.10 (t, J=5.5 Hz, 1H), 8.06 (d, J=8.5 Hz, 2H), 7.44 (d, J=8.1 Hz, 2H), 7.35 (d, J=1.6 Hz, 1H), 7.26 (d, J=1.6 Hz, 1H), 7.24 (d, J=1.6 Hz, 1H), 7.10 (d, J=1.7 Hz, 1H), 7.05 (d, J=1.7 Hz, 1H), 6.85 (d, J=1.6 Hz, 1H), 6.73 (t, J=5.5 Hz, 1H), 4.40 (t, J=5.4 Hz, 2H), 3.88 (s, 3H), 3.86 (s, 3H), 3.60 (t, J=5.5 Hz, 2H), 3.34 (t, J=6.2 Hz, 2H), 3.19 (dd, J=12.7, 6.7 Hz, 2H), 3.04 (dd, J=11.8, 5.9 Hz, 2H), 2.24 (t, J=7.1 Hz, 2H), 2.13 (s, 6H), 1.61 (quintet, J=7.0 Hz, 2H), 1.36 (s, 9H). ¹³C NMR (101 MHz, DMSO) δ162.5, 161.2, 158.5, 158.4, 155.6, 136.2, 130.2, 128.3, 126.5, 123.2, 123.1, 122.8, 122.5, 122.2, 122.1, 121.8, 120.4, 118.9, 118.5, 117.4, 104.8, 104.7, 104.5, 77.6, 70.4, 69.0, 57.1, 47.4, 45.2, 37.1, 36.2, 36.1, 28.2, 27.2. LC-MS: 25.61 min. HRMS (ES+): calculated for C₄₀H₅₀F₃N₁₁O₇ [M+H]+: 854.3919 Found: 854.3943.

Rhodamine B derivative (100 mg, 0.178 mmol), DSC (50.0 mg, 0.195 mmol) and triethylamine (74.2 μL, 0.532 mmol) were mixed volume of DMF was 1.5 mL. At the same time, intermediate compound 5 (181.6 mg, 0.213 mmol) was dissolved in DCM/TFA (50/50, 0.8 mL). After deprotection and evaporation of the solvent, the resulting crude amine was dissolved in 1 mL DMF and neutralized with 0.5 mL triethylamine. Reagent 10 was obtained after purification by column chromatography (silica, DCM/MeOH/NH₄OH, 6/3/1) as a deep purple foam with gold metallic luster (149.1 mg) in 64% yield. LC-MS: 22.29 min. HRMS (ES+): calculated for C₆₇H₇₈F₃N₁₄O₈⁺ M+: 1263.6073 Found: 1263.6062.

### Example 3

Example procedure for the preparation of a reagent used in the invention Following procedures of Chenoweth et al. (J. AM. CHEM. SOC. 2009, 131, 7175-7181) and in line with procedures of Example 2, Reagent 11 is synthesized according to the presented scheme and isolated as a solid.

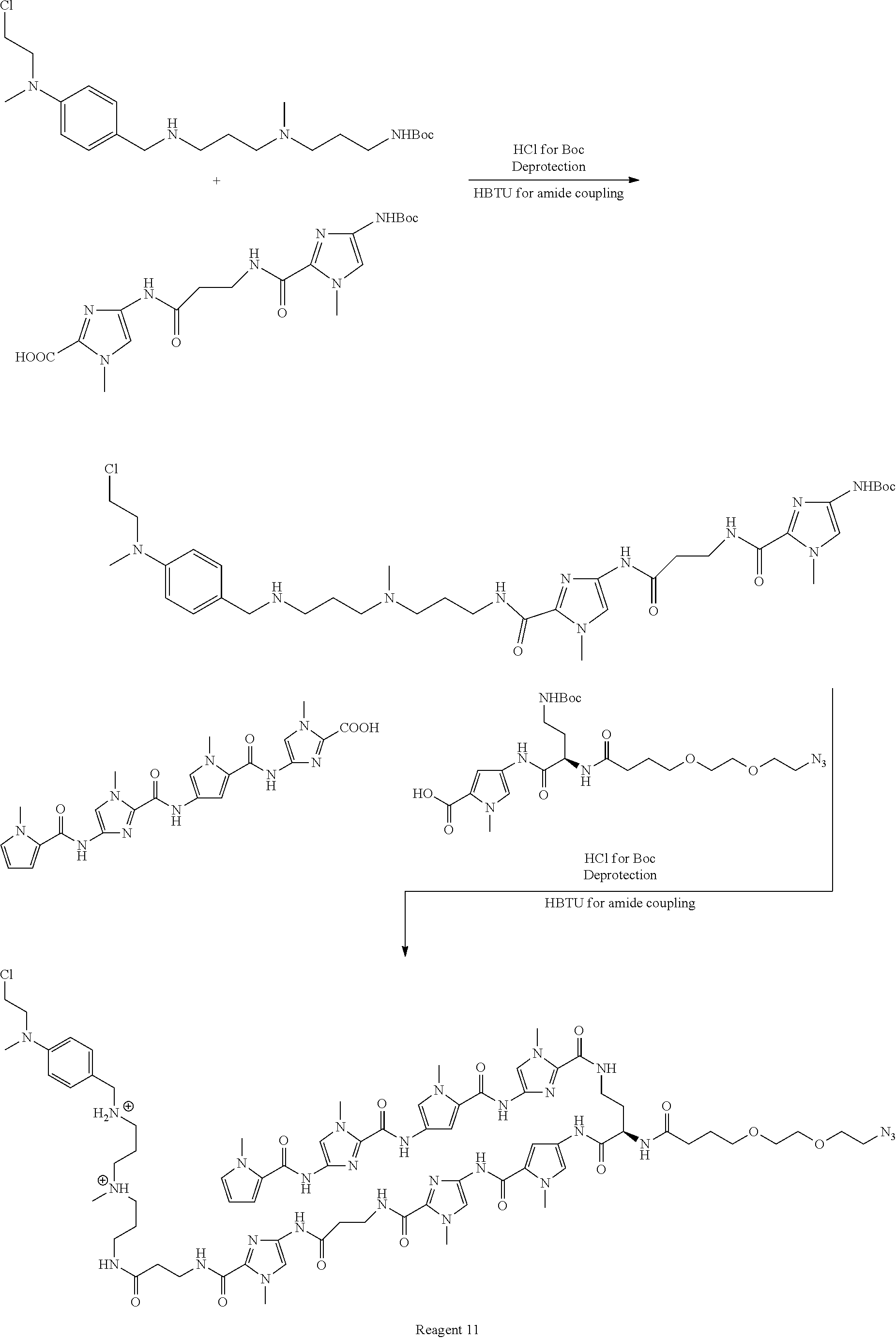

### Example 4

Example of a genomic mapping experiment using reagents and methods of the invention: T7 bacteriophage DNA (1 microgram) was incubated with Reagent 5 for 15 min. at 50° C. in MilliQ, followed by 30 min in a UV-reactor (wavelength of 366 nm) at rt. After covalent DNA labeling, the samples were purified through Chroma spin+TE-1000 columns, and were subsequently stretched on Zeonex coated cover slides (Deen et al, ACS Nano 2015).). The Sequence specific intensity profile was analysed through fluorescence microscopy (Bouwens et al. NAR Genomics and Bioinformatics, Volume 2, Issue 1, March 2020, lqz007), indicating correct assignment of the DNA to its origin.

### Example 5

Example of a genomic mapping experiment using reagents and methods of the invention: T7 bacteriophage DNA (1 microgram) was incubated with Reagent 5 for 15 min. at 50° C. in MilliQ, followed by 30 min in a UV-reactor (wavelength of 366 nm) at rt. After covalent DNA labeling, the samples were purified through Chroma spin+TE-1000 columns, and were subsequently stretched on Zeonex coated cover slides (Deen et al, ACS Nano 2015). The Sequence specific intensity profile was analysed through fluorescence microscopy (Bouwens et al. NAR Genomics and Bioinformatics, Volume 2, Issue 1, March 2020, lqz007). The DNA was incubated at increasing concentrations of competing agent (formamide), but owing to the covalent attachment of the dye, the sequence specifc signal remains.

### REFERENCES

Adey, A., et al. (2014) “In vitro, long-range sequence information for de novo genome assembly via transposase contiguity.” Genome Research 24(12): 2041-2049

Kuleshov, V., et al. (2014) “Whole-genome haplotyping using long reads and statistical methods.” Nature Biotechnology 32(3): 261-266

Voskoboynik, A., et al. (2013) “The genome sequence of the colonial chordate, Botryllus schlosseri.” Elife 2(e00569)

Chaisson, M. J. P., et al. (2015) “Resolving the complexity of the human genome using single-molecule sequencing.” Nature 517(7536): 608-611

Samad, A., et al. (1995) “Optical Mapping—A novel, single-molecule approach to genomic analysis.” Genome Research 5(1): 1-4

Teague, B., et al. (2010) “High-resolution human genome structure by single-molecule analysis.” Proceedings of the National Academy of Sciences of the United States of America 107(24): 10848-10853).

Hastie, A. R., et al. (2013). “Rapid Genome Mapping in Nanochannel Arrays for Highly Complete and Accurate De Novo Sequence Assembly of the Complex Aegilops tauschii Genome.” Plos One 8(2);

Lam, E. T., et al. (2012) “Genome mapping on nanochannel arrays for structural variation analysis and sequence assembly.” Nature Biotechnology 30(8): 771-776

Feuk, L., et al. (2006). “Structural variation in the human genome.” Nature Reviews Genetics 7(2): 85-97

McCaffrey, I, et al. (2016) CRISPR-CAS9 D10A nickase target-specific fluorescent labeling of double strand DNA for whole genome mapping and structural variation analysis. Nucleic Acids Research, 44(2)

Tawar Akash K. J;, et al, (2003), “Minor Groove Binding DNA Ligands with Expanded A/T Sequence Length Recognition, Selective Binding to Bent DNA Regions and Enhanced Fluorescent Properties” Biochemistry 2003, 42, 45, 13339-13346

Akash K. J., et al. (2010) “Groove Binding Ligands for the Interaction with Parallel-Stranded ps-Duplex DNA and Triplex DNA”, Bioconjugate Chemistry. 21, 8, 1389-1403

Kanwal R., (2016) “Dietary Flavones as Dual Inhibitors of DNA Methyltransferases and Histone Methyltransferases” PLoS One. 2016; 11(9): e0162956.

Singh M. et al, (2013), “Bi and tri-substituted phenyl rings containing bisbenzimidazoles bind differentially with DNA duplexes: a biophysical and molecular simulation study”. Molecular BioSystems 2013, 9 (10) , 2541. DOI: 10.1039/c3mb70169g.

Chen Y., et al. (1993) “DNA minor groove-binding ligands: a different class of mammalian DNA topoisomerase I inhibitors” Proceedings of the National Academy of Sciences, 90(17): 8131-8135.

J. Gonzalez-Garcia, et al. (2017) “Supramolecular Principles for Small Molecule Binding to DNA Structures”, 39-70.

Nelson S. M., et al. (2007), “Non-covalent ligand/DNA interactions: Minor groove binding agents Mutation Research/Fundamental and Molecular Mechanisms of Mutagenesis”, 623, 24-40

Compounds and processes for single-pot attachment of label to nucleic acid, US2006/0188927

Proudnikov D., et al, (1996), Chemical methods of DNA and RNA fluorescent labeling, Nucleic Acids Research, Vol. 24, 4535-4532

Prakash A. S., et al., (1990) “DNA-Directed Alkylating Ligands as Potential Antitumor Agents: Sequence Specificity of Alkylation by Intercalating Aniline Mustards”, Biochemistry, 29, 9799-9807

Gottfried A. et al. “Sequence-specific covalent labelling of DNA”, Biochemical Society Transactions, 39(2), 623-628

Kissinger K., et al. “Molecular Recognition between Oligopeptides and Nucleic Acids. Monocationic Imidazole Lexitropsins That Display Enhanced GC Sequence Dependent DNA Binding”, Biochemistry 1987, 26, 5590-5595

Compositions and methods using platinum compounds for nucleic acid labeling: U.S. Pat. No. 6,825,330 B2

Biomolecular labeling, U.S. Pat. No. 6,657,052 B1

Selection of single nucleic acids based on optical signature, US2014/0011686

Methods of specifically labeling nucleic acids using CRISPR/CAS, US 2016/0168621

Belousov E., (1997) “Sequence-specific targeting and covalent modification of human genomic DNA”, Nucleic Acids Research, 25(17), 3440-3444

Methods and devices for single-molecule whole genome analysis U.S. Pat. No. 8,628,919

Geron-Landre, B. et al. (2003) Sequence-specific fluorescent labeling of double-stranded DNA observed at the single molecule level. Nucleic Acids Res. 31, e125 (2003)

Roulon T. (2002) “Coupling of a targeting peptide to plasmid DNA using a new type of padlock oligonucleotide” Bioconjugate. Chemistry. 13, 1134-1139 (2002);

Pfannschmidt C., (1996), Sequence-specific labeling of superhelical DNA by triple helix formation and psoralen crosslinking. Nucleic Acids Res. 24, 1702-1709 (1996).

1. A genomic analysis method, comprising;
Subjecting a polynucleotide to a covalent sequence specific labeling,
Linearizing said sequence specific labeled polynucleotide, and
Obtaining positional information on the sequence specific labels

2. The genomic analysis method according to claim 1, wherein the step of subjecting the polynucleotide to a covalent sequence specific labeling, comprises contacting said polynucleotide with a specific labeling agent comprising a portion, e.g. a binding sequence or sequence specific structure, complementary to a target sequence in the polynucleotide, and wherein the specific labeling agent is configured to bind a label on the polynucleotide at a location within or adjacent to the target sequence.

3. The genomic analysis method according to claim 2, wherein the specific labeling agent comprises a moiety capable of recognizing specific sequences of nucleic acids or abundances of nucleic acids or nucleic acid combinations.

4. The genomic analysis method according to claim 2, wherein the specific labeling agent contains a reactive group which can react covalently with the polynucleotide within or adjacent to the target sequence.

5. The genomic analysis method according to claim 2, wherein the specific labeling agent comprises a label or a reactive labeling group which can react with a label after covalent attachment of the specific labeling agent to the polynucleotide.

6. The genomic analysis method according to claim 2, wherein the binding sequence or sequence specific structure is selected from the group comprising: benzimidazole dimers and oligomers, pyrrole oligomers, flavones, pyrrole-imidazole oligoamides, synthetic oligodeoxynucleotides (ODN), triple-helix forming oligonucleotides, or a combination thereof.

7. The genomic analysis method according to claim 3, wherein the reactive group is selected from the group comprising: platinum complexes, electrophiles (such as mustards, aziridines), nitrenes, carbenes and the like.

8. The genomic analysis method according to claim 5, wherein the label is selected from the group comprising: a fluorophore, a quantum dot, a dendrimer, a nanowire, a bead, a hapten, a streptavidin, an avidin, a neutravidin, a biotin, a reactive group, a peptide, a protein, a magnetic bead, a radiolabel, a non-optical label, or a combination of two or more of the listed items.

9. The genomic analysis method according to claim 5 wherein the reactive labeling groups are bioorthogonal in reactivity.

10. The genomic analysis method according to any one of claims 1-9, wherein the step of linearizing said sequence specific labeled polynucleotide, comprises linearizing the labeled polynucleotide in a fluidic channel, on a surface, or through a nanopore.

11. The genomic analysis method according to claim 2, wherein the polynucleotide is contacted with multiple sequence specific labeling agents, each agent having a portion complementary to a different target sequence in the polynucleotide.

12. The genomic analysis method according to any one of the previous claims wherein the polynucleotide is selected from the list comprising: genomic DNA, plasmid DNA, mRNA, tRNA and genomic RNA; in particular genomic DNA.

13. Use of the genomic analysis method according to any one of the previous claims in providing a barcode of a portion of genomic DNA.
